(19) United States
(12) Patent Application Publication
(10) Pub. No.: US 2007/0224452 A1
(43) Pub. Date: Sep. 27, 2007

(54) Lubricant, magnetic recording medium and head slider

(75) Inventors: Masaaki Sasa, Kawasaki (JP); Hiroshi Chiba, Kawasaki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: FUJITSU LIMITED, Kawasaki (JP)

(21) Appl. No.: 11/515,201

(22) Filed: Sep. 5, 2006

(30) Foreign Application Priority Data
Mar. 24, 2006 (JP) 2006-083137

(51) Int. Cl. G11B 5/33; G11B 5/65; C07C 43/12

(52) U.S. Cl. 428/814; 428/835.8; 568/677

(57) ABSTRACT

A lubricant is provided that can form a sufficiently thin one-molecule film surface even when it is highly polymerized. When used for a magnetic recording medium lubricant layer and/or a head slider lubricant layer of a magnetic recording device, it can make the film thickness of the lubricant layers very small, with the result that it can furnish an increased reliability to the magnetic recording device in a wide temperature-range environment without damaging the flying stability. This lubricant comprises a fluorine-containing polymer having a specific structure. Such a fluorine-containing polymer can be manufactured by reacting a compound having a specific structure and 1,3-butadiene diepoxide.

### CROSS-REFERENCE TO RELATED APPLICATIONS

This application is based upon and claims the benefit of priority from the prior Japanese Patent Application No. 2006-083137, filed on Mar. 24, 2006, the entire contents of which are incorporated herein by reference.

### BACKGROUND OF THE INVENTION

1. Field of the Invention

The present invention relates to a lubricant for magnetic recording devices.

2. Description of the Related Art

In a magnetic recording device, reading and writing of information is carried out while a head slider having a magnetic recording transducer (in the present invention, also referred to simply as a ‘head’) flies over a hard disk which is a magnetic recording medium.

The distance between the head and a magnetic layer for recording (writing) or reproducing (reading) magnetic information on the hard disk is known as the magnetic spacing; the smaller the magnetic spacing, the greater the recording density. Meanwhile, to increase the information transfer rate, the rotational speed of the hard disk must be made high. In recent years, as the recording density and the transfer rate have been increased, there has been progress in reducing the flying height (also called floating height) and increasing in the rotational speed; currently, the head flying height has become approximately 10 nm, and the rotational speed has become approximately 15,000 rpm.

With a hard disk drive, to increase the reliability of the drive, a lubricant is applied, in a thickness of approximately 1 to 2 nm, onto the magnetic disk or the head slider, in general. This lubricant reduces friction and wear upon contact of the head with the disk, preventing the occurrence of faults.

The film thickness of the lubricant accounts for approximately 10% of the head flying height, and hence it cannot be disregarded as a factor for the magnetic spacing (see, for example, X. Ma et al., I.E.E.E. Trans. Magn., 2001, Vol. 37, p. 1824). Consequently, to improve the recording density, it is becoming important to make the lubricant film thickness smaller so as to decrease the magnetic spacing.

### SUMMARY OF THE INVENTION

However, since lubricants have a finite molecular size, it is not possible to reduce the lubricant film thickness less than the one-molecule film thickness. Although, it may be possible to make it thinner than that on an average, it will result in a decreased lubricant coverage.

It is known that the one-molecule film thickness of a lubricant is determined by the molecular weight of the lubricant (see, for example, X. Ma et al., Journal of Chemical Physics, 1999, Vol. 110, p. 3129 to 3,137). Accordingly, it is possible to reduce the one-molecule film thickness by reducing the molecular weight.

However, a lubricant with a smaller molecular weight tends to be evaporated. Such a lubricant also has a property of easy scattering at a high-speed rotation. Accordingly, there is a limit, at present, on the movement towards a lower molecular weight, in view of the HDI (Head Disk Interface) properties such as depletion or loss of the lubricant at a high rotation speed. To go over the limit, it is necessary to have a lubricant that gives a sufficiently small one-molecule film thickness even when highly polymerized.

It is an object of the present invention to solve the above-described problems, and to develop a technology that can provide a very thin lubricant layer even when a lubricant with a high molecular weight is used, and can increase the reliability in a wide temperature-range environment without damaging the flying stability. Other objects and advantages of the present invention will be apparent from the following detailed description.

According to one aspect of the present invention, provided is a lubricant comprising a fluorine-containing polymer having a structure represented by formula 1,

H—{(XZ)_{m′}(YZ)ₙ}-(X_{δ},Y_{(1-δ)})—H (1)

[in formula 1, m and n are each a real number not less than zero (wherein m and n are not zero at the same time); {(XZ)ₘ, (YZ)ₙ} means that the structure unit XZ and the structure unit YZ may each take a random sequence or a blocked sequence; similarly, (X_{δ}, Y_{(1-δ)}) means that the structure unit X and the structure unit Y may each take a random sequence or a blocked sequence; δ is a real number not less than zero and not more than 1; and the chemical structures of X, Y and Z are as shown in formulae 12, 13 and 14 in this order, wherein hydrogens on X, Y and Z may be each substituted with an organic group having from one to three carbons that may contain an ether bond, and may have its hydrogen or hydrogens substituted with one or both of a polar group and fluorine,]

—O(CH₂CH₂O)ₐCH₂CF₂O(CF₂CF₂O)ₚ(CF₂O)_{q}CF₂CH₂ (OCH₂CH₂)_{b}O— (12)

{in formula 12, a, b, p and q are each a real number not less than zero (wherein p and q are not zero at the same time); and the structure units CF₂CF₂O and CF₂O may each take a random sequence or a blocked sequence,}

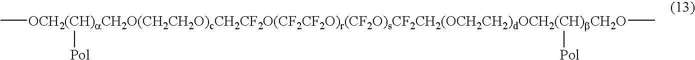

{in formula 13, c, d, r and s are each a real number not less than zero (wherein r and s are not zero at the same time); α and β are, independently from each other, a positive real number; and the structure units CF₂CF₂O and CF₂O may each take a random sequence or a blocked sequence; and Pols are, independently from each other and independently from the other formulae, a polar group,}

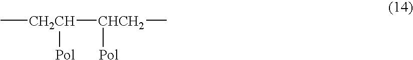

(in formula 14, Pols are, independently from each other and independently from the other formulae, a polar group).

By this aspect, it is possible to obtain a lubricant that gives a sufficiently small one-molecule film thickness even when it has a high molecular weight. Using the lubricant, it is possible to make the film thickness of a lubricant layer very small, even when it has a high molecular weight, and can increase the reliability in a wide temperature-range environment, without damaging the flying stability.

Preferable are that the number average molecular weight of molecular chains that are each between two adjacent polar groups of the fluorine-containing polymer is not less than 500; that the number average molecular weight of the fluorine-containing polymer is not less than 2,000 and not more than 12,000; that the Pol is a hydroxy group; that the number average molecular weight of molecular chains that are each between two adjacent polar groups of the fluorine-containing polymer is not more than 3,000; that the fluorine-containing polymer has a structure represented by formula 3 at its molecular end,

—R (3)

(in formula 3, R represents a polar group or a hydrocarbon group);
that part or the whole of structure units C₂F₄O and CF₂O in formula 1 is substituted with a structure represented by formula 4 or a structure represented by formula 5,

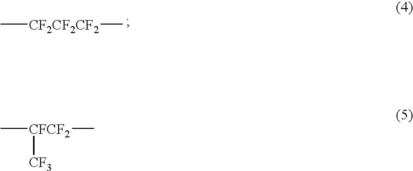

that no polar group is present except a hydroxy group in the fluorine-containing polymer; and that there are from 1.0 to 10.0 hydroxy groups per molecule on an average in the intermediate section of a molecular chain of the fluorine-containing polymer.

According to another aspect of the present invention, provided is a lubricant comprising a fluorine-containing polymer obtained by reacting a compound having a structure represented by formula 2 and 1,3-butadiene diepoxide,

HO(CH₂CH₂O)ₓCH₂CF₂O(CF₂CF₂O)_{p″}(CF₂O)_{q″}CF₂CH₂(OCH₂CH₂)_{y}OH (2)

(in formula 2, p″, q″, x and y are, independently from each other, a real number not less than zero (wherein p″ and q″ are not zero at the same time); and the structure units CF₂CF₂O and CF₂O may each take a random sequence or a blocked sequence.)

By this aspect, it is also possible to obtain a lubricant that gives a sufficiently small one-molecule film thickness even when it has a high molecular weight. Using the lubricant, it is possible to make the film thickness of a lubricant layer very small, even when it has a high molecular weight, and can increase the reliability in a wide temperature-range environment, without damaging the flying stability.

Preferable are that the fluorine-containing polymer is obtained by reacting a compound having a structure represented by formula 2 and 1,3-butadiene diepoxide followed by a further reaction with glycidol; that the number average molecular weight of the fluorine-containing polymer is not less than 2,000 and not more than 12,000; that there are from 1.0 to 10.0 hydroxy groups per molecule on an average in the intermediate section of a molecular chain of the fluorine-containing polymer; that the fluorine-containing polymer has a structure represented by formula 3 at its molecular end,

—R (3)

(in formula 3, R represents a polar group or a hydrocarbon group);
that part or the whole of structure units C₂F₄O and CF₂O in formula 2 is substituted with a structure represented by formula 4 or a structure represented by formula 5,

—CF₂CF₂CF₂— (4); and

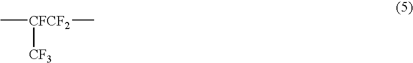

that the lubricant comprises a fluorine-containing polymer other than the fluorine-containing polymer; that the fluorine-containing polymer other than the fluorine-containing polymer is a compound having at least one structure selected from the group consisting of formulae 9, 10 and 11,

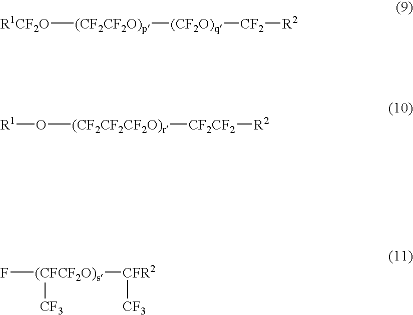

(in formulae 9 to 11, R¹ and R² are, independently from each other and independently in each formula, a group selected from the group consisting of a hydroxy group, a carboxy group, an amino group and a phosphazene ring, a monovalent aliphatic hydrocarbon group that has, as a substituent group or groups, one or more groups selected from the group consisting of a hydroxy group, a carboxy group, an amino group and a phosphazene ring, may have a carbonyl group, or an ether group, or a carbonyl group and an ether group, may have a double bond, or a triple bond, or a double bond and a triple bond, and may be branched, and a monovalent aromatic hydrocarbon group and a monovalent heterocyclic aromatic hydrocarbon group that comprise, as a substituent group or groups, one or more groups selected from the group consisting of a hydroxy group, a carboxy group, an amino group and a phosphazene ring; p′, q′, r′ and s′ are each a positive real number; and each structure unit of formulae 9, 10 and 11 may take a random sequence or a blocked sequence in the structure for each compound).

According to still other aspects of the present invention, provided are a magnetic recording medium comprising a magnetic layer, a protective layer over the magnetic layer and a magnetic recording medium lubricant layer on the protective layer, the magnetic recording medium lubricant layer having been formed by the application of the above-described lubricant, and a head slider having a recording transducer for recording to and/or reproducing information from a magnetic recording medium, comprising a protective layer and a head slider lubricant layer having been formed by the application of the above-described lubricant, over the surface of the head slider that faces the magnetic recording medium.

By these aspects, it is possible to obtain a magnetic recording medium, a head slider and a magnetic recording device with a very thin lubricant layer, and that can increase the reliability in a wide temperature-range environment, without damaging the flying stability.

By the present invention, a lubricant is obtained which gives a sufficiently small one-molecule film thickness even when it has a high molecular weight. Applying this lubricant to a magnetic recording medium lubricant layer and/or a head slider lubricant layer of a magnetic recording device, it is possible to make the film thickness of the lubricant layers very small and increase the reliability of the magnetic recording device in a wide temperature-range environment, without damaging the flying stability.

### BRIEF DESCRIPTION OF THE DRAWINGS

FIG. 1 is a schematic view showing a state in which a fluorine-containing polymer is adhered to a coating surface with polar groups Pols;

FIG. 2 is another schematic view showing a state in which a fluorine-containing polymer is adhered to a coating surface with polar groups Pols;

FIG. 3 is another schematic view showing a state in which a fluorine-containing polymer is adhered to a coating surface with polar groups Pols;

FIG. 4 is a view for explaining how to calculate the number average molecular weight of molecular chains that are each between two adjacent polar groups;

FIG. 5 is a ¹HNMR chart of a compound obtained in EXAMPLE 1;

FIG. 6 is a ¹³C NMR chart of a compound obtained in EXAMPLE 1;

FIG. 7 is a ¹⁹F NMR chart of a compound obtained in EXAMPLE 1;

FIG. 8 is a graph showing the relationship between the number average molecular weight (Mc) of structure parts between two adjacent hydroxy groups, and the one-molecule layer film thickness;

FIG. 9 is a chart showing a glide test result;

FIG. 10 is another chart showing a glide test result;

FIG. 11 is a chart showing TDV, TOV test results;

FIG. 12 is another chart showing TDV, TOV test results;

FIG. 13 is a schematic plan view showing the internal structure of a hard disk apparatus; and

FIG. 14 is a schematic side cross-sectional view showing the relationship between a head and a magnetic recording medium of a hard disk apparatus.

### DESCRIPTION OF THE PREFERRED EMBODIMENTS

Following is a description of embodiments of the present invention through drawings, tables, formulae, examples and so on. Note that these drawings, tables, formulae, examples and so on, as well as the explanation merely illustrate the present invention, and do not limit the scope of the present invention. It goes without saying that the other embodiments can also come under the category of the present invention to the extent that the gist of the present invention is conformed to.

As a lubricant which has a molecular structure that does not give a larger one-molecule film thickness when it has a larger molecular weight, perfluoropolyethers are known that have polar groups in the intermediate section of a molecule (see Japanese Unexamined Patent Application Publication No. 2003-162810, claims, for example). However, the specific molecular structure, the molecular weight range, etc. remain unknown.

As a result of investigation, it was found that a fluorine-containing polymer that is not a perfluoro structure, and has a specific structure provides a lubricant layer with a very small film thickness even when a lubricant with a high molecular weight is used, when it is used for a magnetic recording medium lubricant layer and/or a head slider lubricant layer of a magnetic recording device, and can provide an increased reliability to the magnetic recording device in a wide-temperature range environment without damaging the flying stability.

In other words, a lubricant according to the present invention comprises a fluorine-containing polymer obtained by reacting a compound having a structure represented by formula 2 and 1,3-butadiene diepoxide,

HO(CH₂CH₂O)ₓCH₂CF₂O(CF₂CF₂O)_{p″}(CF₂O)_{q″}CF₂CH₂(OCH₂CH₂)_{y}OH (2)

(in formula 2, p″, q″, x and y are, independently from each other, a real number not less than zero (wherein p″ and q″ are not zero at the same time); and the structure units CF₂CF₂O and CF₂O may each take a random sequence or a blocked sequence.)

In the present invention including the following, when a symbol represented as a subscript indicates a “real number”, the “real number”, refers to an average value for the part or compound having the structure.

Any type of known methods may be employed for the above-described reaction. This fluorine-containing polymer may have passed through other reactions and steps of purification in addition to the reaction described above. The hydroxy group or groups in the intermediate section of a molecular chain derive from the 1,3-butadiene diepoxide. Most of the terminal structures of molecules are hydroxy groups.

Furthermore, a lubricant according to the present invention may be the above-described fluorine-containing polymer that is obtained by reacting a compound having a structure represented by formula 2 and 1,3-butadiene diepoxide, followed by a reaction with glycidol. In addition to the above-described reactions, other reactions and steps of purification may also be added.

Any type of known methods may be employed for the above-described reaction. The hydroxy group or groups in the intermediate section of a molecular chain derive from the 1,3-butadiene diepoxide. The —CH₂—CH(OH)—CH₂—OH at the ends of a molecular chain derives from the glycidol.

It is also possible to have the thus obtained polymer react with an appropriate compound to substitute or cap the terminal hydroxy groups. Methyl iodide, carboxylic anhydride, etc. may be used for the substitution or capping. By this, the molecular ends of a fluorine-containing polymer come to have a structure represented by formula 3. OCOCH₃, COOH, OCH₃, and OCH₂CH₃ are examples of the polar group R, and an alkyl group is an example of the hydrocarbon group.

—R (3)

(in formula 3, R represents a polar group or a hydrocarbon group).

It is also preferable that a fluorine-containing polymer according to the present invention is obtained by using a compound that is obtained by substituting part or the whole of structure units C₂F₄O and CF₂O in formula 2 with a structure represented by formula 4 or a structure represented by formula 5.

—CF₂CF₂CF₂— (4)

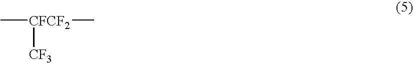

It is to be noted that compounds having the same structure as formula 1 can be synthesized by reacting 1,3-butadiene diepoxide or 1,3-butadiene diepoxide and glycidol with a commercially available fluoropolymer having a structure represented by formula 4 or a structure represented by formula 5.

By the present invention, a lubricant is obtained that can provide a sufficiently small one-molecule film thickness even when it is highly polymerized. In a lubricant comprising a fluorine-containing polymer with such a structure, it is considered that polar groups (hydroxy groups) located in the intermediate section of a molecule are probably adhered to the surface to which the lubricant is applied (called a coating surface hereinafter), together with the polar groups (hydroxy groups) located at or in the vicinity of the molecular ends, so that the molecules will not rise high above the coating surface, and accordingly, this effect results in a lubricant layer with a very small film thickness, even when the lubricant has a high molecular weight.

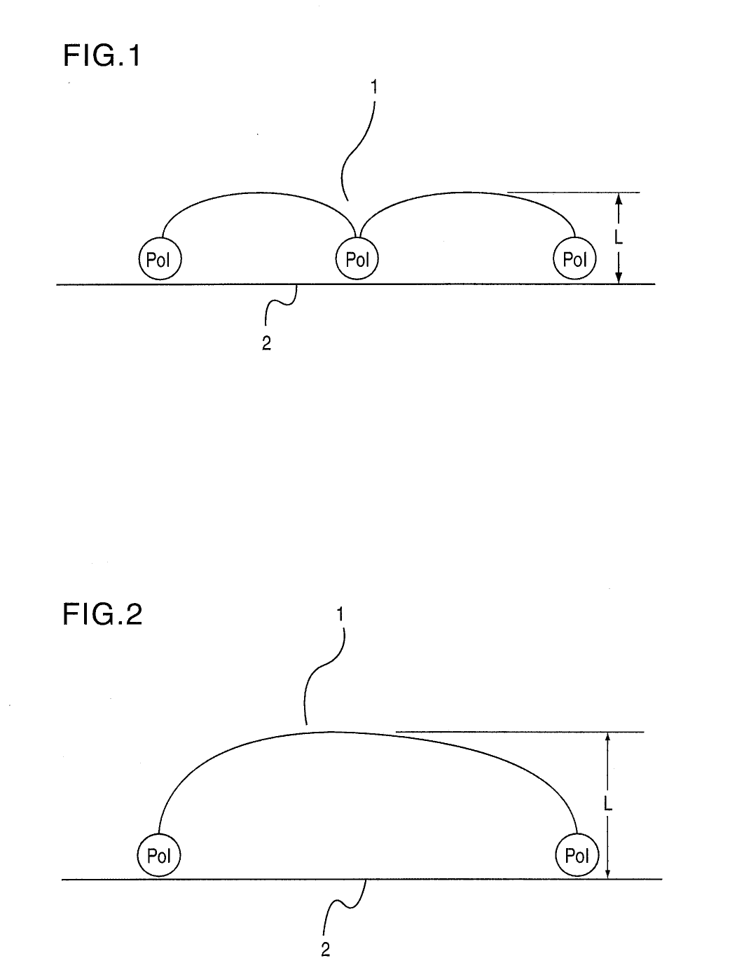

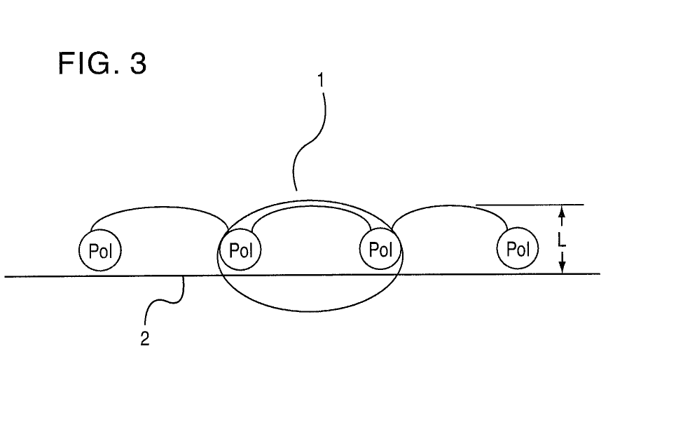

This behavior will be explained with FIGS. 1 to 3. FIG. 2 shows a case of polar groups Pols locating at both ends of a molecule 1 of a fluorine-containing polymer. FIG. 1 shows a case of polar groups Pols locating at both ends, and one polar group Pol in the intermediate section, of a molecule 1 of a fluorine-containing polymer. FIG. 3 schematically shows a case of polar groups Pols locating at both ends, and two polar groups Pols in the intermediate section, of a molecule 1 of a fluorine-containing polymer. Consider that the length of the molecules in FIGS. 1 to 3 are the same. Then, it is understood that the distance L away from the coating surface 2 of a molecule 1 of a fluorine-containing polymer will be smaller when a polar group is present in the intermediate section of a molecular chain.

It is considered that the reason of a large extent of the effect that a lubricant can be obtained that gives a sufficiently small one-molecule film thickness even when it has a high molecular weight, if a fluorine-containing polymer according to the present invention is used, is probably that two adjacent hydroxy groups with two carbon atoms in between are formed in the polymer chain due to the use of 1,3-butadiene diepoxide, and the two adjacent hydroxy groups with two carbon atoms in between are fixed to the coating surface more strongly than lone hydroxy groups.

It is preferable that the number average molecular weight of the fluorine-containing polymer is not less than 2,000 and not more than 12,000. Less than 2,000, degradation of migration properties as shown by the decreasing rates of endurance and film thickness at a high temperature will be observed. There will also be a tendency of easier scattering at a high rotation. Over 12,000, the viscosity will be excessively high, and a tendency will appear towards the degradation of endurance at a low temperature of a magnetic recording medium and/or a head as explained above.

In addition, it is preferable that there are from 1.0 to 10.0 hydroxy groups per molecule on an average in the intermediate section of a molecular chain. Less than 1.0, a thicker one-molecule film thickness tends to result. In a typical case, the one-molecule film thickness may exceed 2 nm. Over 10.0, no particular improvement is obtained. Rather, if the number average molecular weight of a fluorine-containing polymer is in the range of 2,000 to 12,000, it will result in too many hydroxy groups present in a short molecular chain in a typical case, and a problem will be generated that cohesion of molecules tends to occur due to the intermolecular interaction caused by the hydroxy groups, with the result that the lubricant film thickness changes (in other words, the lubricant film thickness increases as time passes by).

If the above-described range is kept, it is possible to consider that, as long as the adhesion to the coating surface is well-established, the distance L by which the fluorine-containing polymer according to the present invention is spaced away from the coating surface is on the same level as the distance L by which a fluorine-containing polymer having polar groups only at the molecular ends, and having, as a whole molecule, a molecular weight that is the same as the molecular weight of the above-described structural part between the polar groups.

It is more preferable that the number of hydroxy groups in the intermediate section of a molecular chain is from 1.0 to 5.0 on an average per molecule. It is still more preferable that the number is from 2.0 to 5.0 on an average per molecule. If at least one 1,3-butadiene diepoxide is incorporated in the intermediate section of each molecule, the number of hydroxy groups in the intermediate section of a molecular chain will be 2 or more, and accordingly, is preferable.

It is to be noted that, if the above-described explanation is further generalized, it is possible to examine the situation from a viewpoint: when there are polar groups in the intermediate section of one molecule, on what level the chemical formula weight of at least one molecular chain is among the molecular chains between polar groups.

As a result of investigation under this way of thinking, it was found that, if the following conditions are satisfied, it is possible to obtain a lubricant that can give a sufficiently small one-molecule film thickness: a first condition is that, in addition to hydroxy group, other polar groups can be appropriately selected from known polar groups such as carboxy group, carbonyl group, sulfonic acid group, nitro group and nitrile group (however, ether bond is not included); a second condition is that the fluorine-containing polymer contained in the lubricant has a structure represented by formula 1,

H—{(XZ)_{m′}(YZ)ₙ}-(X_{δ}, Y_{(1-δ)})—H (1)

[in formula 1, m and n are each a real number not less than zero (wherein m and n are not zero at the same time); {(XZ)ₘ, (YZ)ₙ} means that the structure unit XZ and the structure unit YZ may each take a random sequence or a blocked sequence; similarly, (X_{δ}, Y_{(1-δ)}) means that the structure unit X and the structure unit Y may each take a random sequence or a blocked sequence; δ is a real number not less than zero and not more than 1; and the chemical structures of X, Y and Z are as shown in formulae 12, 13 and 14 in this order, wherein hydrogens on X, Y and Z may be each substituted with an organic group having from one to three carbons that may contain an ether bond, and may have its hydrogen or hydrogens substituted with one or both of a polar group and fluorine,]

—O(CH₂CH₂O)ₐCH₂CF₂O(CF₂CF₂O)ₚ(CF₂O)_{q}CF₂CH₂(OCH₂CH₂)_{b}O— (12)

{in formula 12, a, b, p and q are each a real number not less than zero (wherein p and q are not zero at the same time); and the structure units CF₂CF₂O and CF₂O may each take a random sequence or a blocked sequence,}

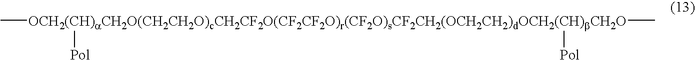

{in formula 13, c, d, r and s are each a real number not less than zero (wherein r and s are not zero at the same time); α and β are, independently from each other, a positive real number; the structure units CF₂CF₂O and CF₂O may each take a random sequence or a blocked sequence; and Pols are, independently from each other and independently from the other formulae, a polar group,}

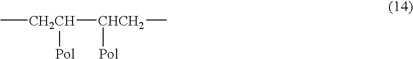

(in formula 14, Pols are, independently from each other and independently from the other formulae, a polar group). It is to be noted that each of the α and β in formula 1 means that there are CH-Pol pairs in the number of α or β. For example, α=2 means CH(Pol)-CH(Pol). In many cases, it is advantageous that the two Pols are the same in formula 14 from the viewpoint of ease of the production.

Regarding the fluorine-containing polymer according to the present invention, it is preferable that the number average molecular weight of the fluorine-containing polymer is not less than 2,000 and not more than 12,000, and that the number average molecular weight of molecular chains that are each between two adjacent polar groups is not less than 500. The upper limit and lower limit of the number average molecular weight are based on the same reasons as described above.

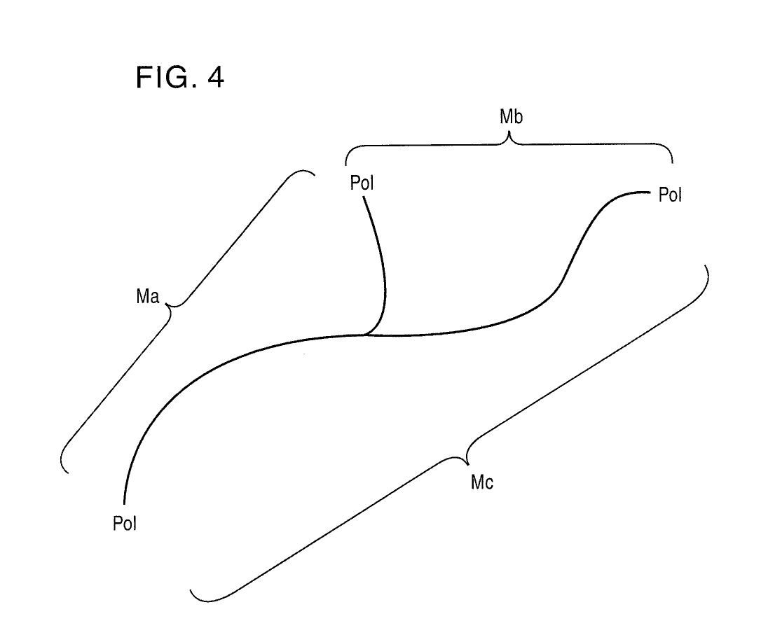

Polar groups themselves are not included in the calculation of the number average molecular weight of molecular chains that are each between two adjacent polar groups. In the case of two adjacent polar groups without a branched structure, it is sufficient to determine the number average molecular weight of molecular chains that are each between the two adjacent polar groups on the polymer chain. In the case of two adjacent polar groups with a branched structure which has no polar groups, the molecular weight is counted for the part without the branched structure. In the case of two adjacent polar groups with a branched structure which has a polar group, the molecular weight of molecular chains that are each between two adjacent polar groups when seen from the polar group in the branched structure is also counted. In this case, the molecular weight is counted without including the structural part branching from the chain part connecting the two adjacent polar groups. In the case of FIG. 4, for example, Ma, Mb and Mc are counted. In FIG. 4, a Pol refers to a polar group. In the present invention, the term “adjacent” means the relationship to the next polar group on a polymer chain (including a molecular chain), as shown in FIG. 4.

It is to be noted that, in the case of a polar group at a molecular end and a polar group in the vicinity are present close to each other, if the number average molecular weight is counted also for the molecular chain between such a polar group at a molecular end and a polar group in the vicinity, there would be cases that the molecular weights of molecular chains between two adjacent polar groups has a large variation, and accordingly, the above-described condition of not less than 500 is not enough. As a result of investigation, it was found that, when there are two or more polar groups (including the terminal polar group) in an area of five carbons from the end of a molecule, it is preferable to bring together those polar groups in the area of five carbons from the molecular end to treat them as one polar group, and then select a fluorine-containing polymer to satisfy the condition of not less than 500. Also, in the case that polar groups are present extremely close to each other just like a case of butadiene diepoxide, it is preferable to bring together the two adjacent polar groups sandwiching two carbon atoms to treat them as one polar group, and then select a fluorine-containing polymer to satisfy the condition of not less than 500.

When the number average molecular weight obtained in this way is less than 500, it is difficult to make the film thickness of a lubricant layer very small, probably because cohesion of the polymer tends to occur due to a small distance between polar groups. It is more preferable that the number average molecular weight is not less than 1,000.

The fluorine-containing polymer having a structure represented by formula 1 may consists of one kind of polymer, or may comprise plural kinds of polymers. For example, if n=0 and δ=1, formula 1 provides a structure which has no Y component, and if m=δ=0, formula 1 provides a structure which has no X component. The fluorine-containing polymer according to the present invention may be composed of a fluorine-containing polymer having either one of these structures, or may comprise both structures in a mixed state.

The phrase “hydrogens on X, Y and Z may be each substituted with an organic group having from one to three carbons that may contain an ether bond, and may have its hydrogen or hydrogens substituted with one or both of a polar group and fluorine” means that the structure represented by formula 1 may be not only a “structure without branching” that is shown from formula 1 itself, but also a structure with branching; that the branching may contain from one to three carbons, and oxygen may be present between carbons; and that, in such a case, hydrogens bonded to a carbon may be substituted with a polar group and/or fluorine. The above-described Pols are independently from each other in each formula and also independently between formulae 13 and 14, a polar group. In the present invention, the “polar group” means a polar group that does not contain an ether bond as described above, and hydroxy group, carboxy group, carbonyl group, sulfonic acid group, nitro group and nitrile group are the examples. Generally speaking, hydroxy group is preferable as the polar group, from the viewpoints of ease of procurement, effects on the system such as corrosion, etc. In this meaning, it is more preferable that the polar group consists only of hydroxy groups. Also, from the viewpoint of ease of production, it is preferable in many cases that the fluorine-containing polymer does not have a polar group other than Pols in formulae 13 and 14, and that it does not have a branched structure.

As explained in the examples, it is preferable in any of the above-described cases that the number average molecular weight of molecular chains that are each between two adjacent polar groups of the fluorine-containing polymer is not more than 3,000. It has been found that, if the molecular weight of the structure parts between two adjacent polar groups when seen from the viewpoint of an average value exceeds 3,000, the film thickness of the lubricant becomes large, and accordingly it is preferably not more than 3,000.

Regarding a fluorine-containing polymer having such a structure, the end group may also have a structure represented by formula 3 in the same way as in the above-described case. The structure R in formula 3 may be introduced by any known methods.

Furthermore, it is also preferable in formula 1 that part or the whole of structure units C₂F₄O and CF₂O is substituted with a structure represented by formula 4 or a structure represented by formula 5. It is expected that the presence of such structures will reduce the surface free energy.

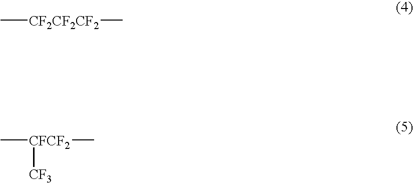

The above-described structures of formulae 3, 4 and 5 may be introduced by any known methods.

For the above-described fluorine-containing polymer, it may be possible to employ, together with the condition of a number average molecular weight of not less than 500, or instead of the condition, a condition that there are from 1.0 to 10.0 polar groups (hydroxy groups for example) per molecule on an average in the intermediate section of a molecular chain. It is more preferable that the number of hydroxy groups in the intermediate section of a molecular chain is from 1.0 to 5.0 on an average per molecule. It is still more preferable that the number is from 2.0 to 5.0 on an average per molecule.

A lubricant according to the present invention may consist only of any of the above-described fluorine-containing polymers. It may also comprise another compound. It is preferable for the other compound to be a fluorine-containing polymer other than the above-described fluorine-containing polymer. It is preferable that such a fluorine-containing polymer has properties required for a lubricant regarding the molecular weight, fluorine content, etc. At least one compound selected from the group consisting of those represented by formulae 9, 10 and 11 is preferable as such a fluorine-containing polymer. They are commercially available.

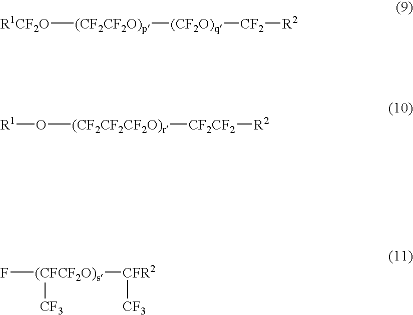

In formulae 9 to 11, R¹ and R² are, independently from each other and independently in each formula, a group selected from the group consisting of a hydroxy group, a carboxy group, an amino group and a phosphazene ring, a monovalent aliphatic hydrocarbon group that has, as a substituent group or groups, one or more groups selected from the group consisting of a hydroxy group, a carboxy group, an amino group and a phosphazene ring, may have a carbonyl group, or an ether group, or a carbonyl group and an ether group, may have a double bond, or a triple bond, or a double bond and a triple bond, and may be branched, and a monovalent aromatic hydrocarbon group and a monovalent heterocyclic aromatic hydrocarbon group that comprise, as a substituent group or groups, one or more groups selected from the group consisting of a hydroxy group, a carboxy group, an amino group and a phosphazene ring; p′, q′, r′ and s′ are each a positive real number; and each structure unit of formulae 9, 10 and 11 may take a random sequence or a blocked sequence in the structure of each compound.

The above-described various lubricants according to the present invention may be favorably used for a lubricant layer installed on a magnetic recording medium or a head slider of a magnetic recording device. In other words, a magnetic recording medium comprising a magnetic layer, a protective layer over the magnetic layer and a magnetic recording medium lubricant layer on the protective layer, the magnetic recording medium lubricant layer having been formed by the application of the above-described lubricant, and a head slider equipped with a head for recording to and/or reproducing information from a magnetic recording medium, comprising a protective layer and a head slider lubricant layer having been formed by the application of the above-described lubricant, over the surface of the head slider that faces the magnetic recording medium, can make the lubricant layers very thin, and can comply with the requirement of a narrow head flying height.

In this case, heat treatment after the application of a lubricant is preferable for both of the magnetic recording medium lubricant layer and head slider lubricant layer, in order to improve the surface uniformity and the adhesion to the coating surface, of the lubricant applied to the surface. The heat treatment is preferably carried out at an ambient temperature in the range of from 70 to 150° C. and for a period of from 0.5 to 2 hours. It is also preferable to carry out UV irradiation treatment after the application of a lubricant. Only one or both of the heat treatment and UV irradiation treatment may be carried out. However, it is preferable to carry out both of them, and perform the UV irradiation treatment after the heat treatment, because it improves the adhesion with the coating surface.

There is no limitation on the material for forming the coating surfaces of a magnetic recording medium and a head slider for applying a lubricant according to the present invention, and may be appropriately selected from known materials. It is generally preferable that the material has an affinity for polar groups such as hydroxy groups to some extent.

A hard disk device will be used in the following to explain the magnetic recording device according to the present invention. However, all and any magnetic recording devices that use a magnetic recording medium and a head slider may be included in the “magnetic recording device” according to the present invention, as long as they are not out of the gist of the present invention.

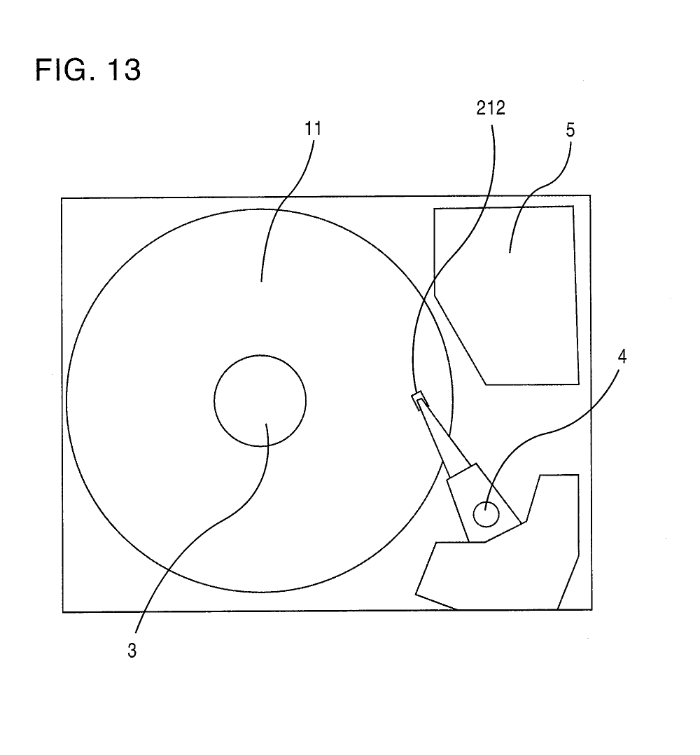

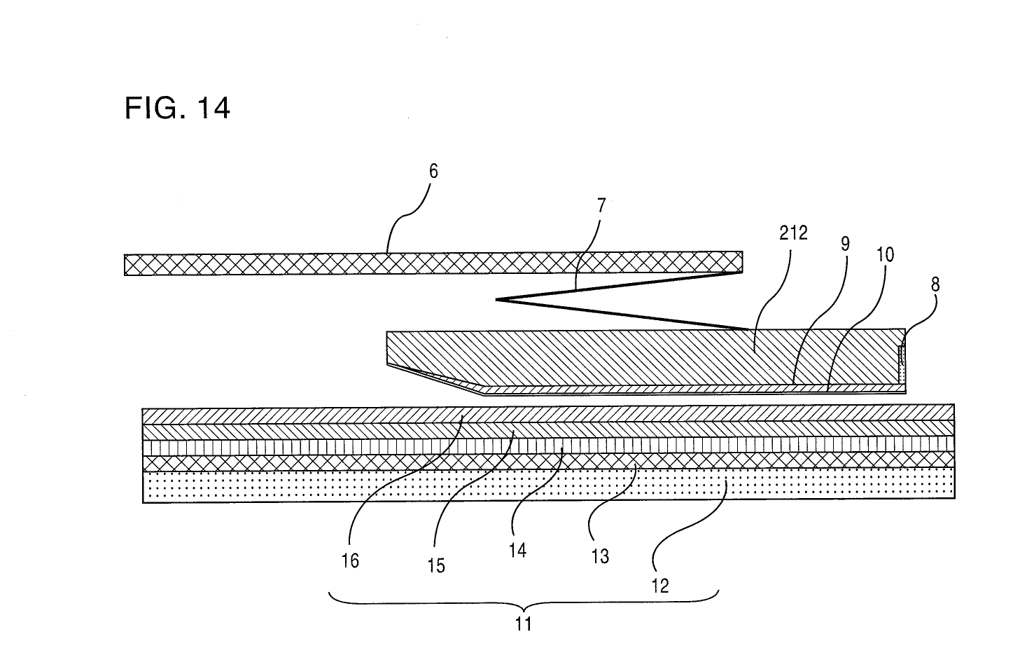

FIG. 13 is a schematic plan view showing the internal structure of a hard disk device, and FIG. 14 is a schematic side cross-sectional view showing the relationship between a head and a magnetic recording medium (a view of a cross-section in a direction perpendicular to the magnetic layer of the magnetic recording medium).

As shown in FIG. 13, this hard disk drive device has, as main components, a magnetic recording medium 11, a head slider 212 having a head, a rotation control mechanism (e.g. a spindle motor) 3 for the magnetic recording medium 11, a head positioning mechanism 4, and recording/reproduction signal processing circuit (read/write amplifier or the like) 5.

As shown in FIG. 14, the head slider 212 is connected to the head positioning mechanism 4 by a suspension 6 and gimbals 7 for flexibly supporting the head slider 212, and a head 8 is provided at the tip of the head slider 212. A head slider protective layer 9 and a head slider lubricant layer 10 are provided on the surface of the head slider.

The magnetic recording medium 11 has, from the bottom in FIG. 14, a substrate 12, a Cr underlayer 13, a magnetic layer 14, a magnetic recording medium protective layer 15, a magnetic recording medium lubricant layer 16 and so on. Other layers such as a seed layer may be provided, but these are omitted from the drawings.

### EXAMPLES

EXAMPLES according to the present invention will be explained in detail as follows.

### Example 1

(Synthesis of a Lubricant)

To a 500 mL acetone, 100 g of a commercially available FOMBLIN Z DOL (a product made by Solvay Solexis, x=y=0 in formula 2, the molecular weight being 2,020), and 0.15 mol of 1,3-butadiene diepoxide were added. To the mixture under good mixing, 0.11 mol of sodium hydroxide in 5 g water was added dropwise for the duration of 10 minutes, followed by heating and refluxing for six hours.

Afterwards, acetone was evaporated with an evaporator, 25 g of trifluoroacetic acid and 250 mL of water were added and stirred at 70° C. for three hours. The deposit was recovered and washed with water at 80° C.

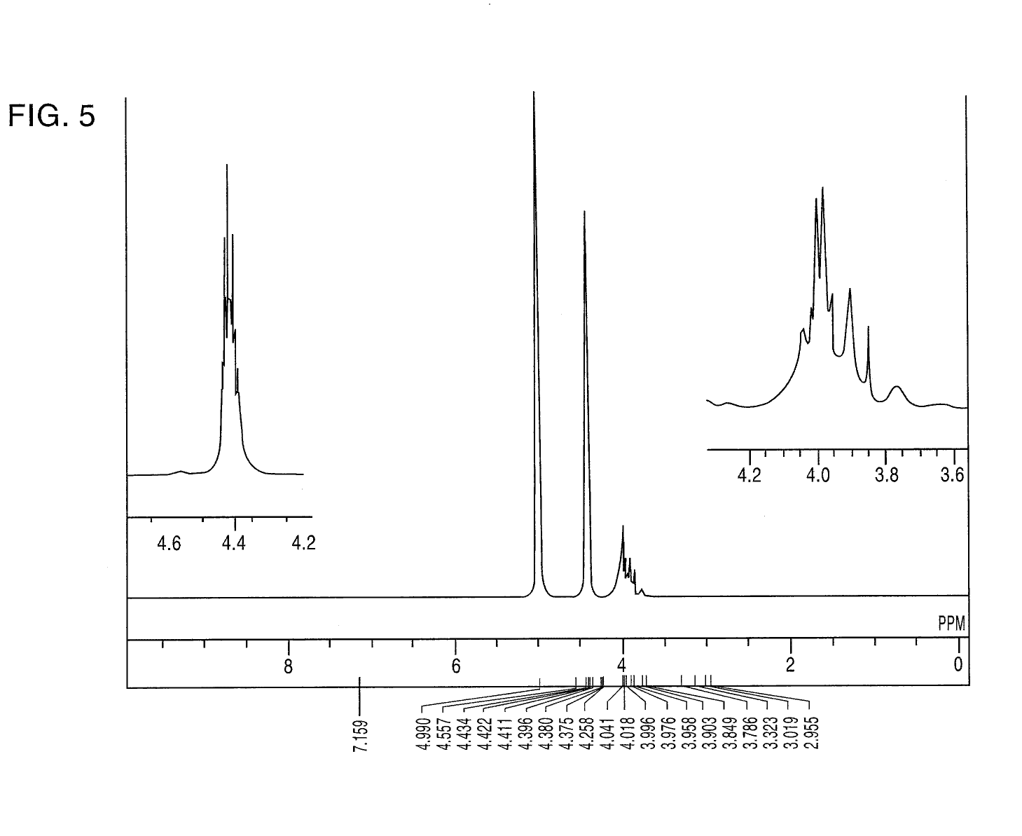

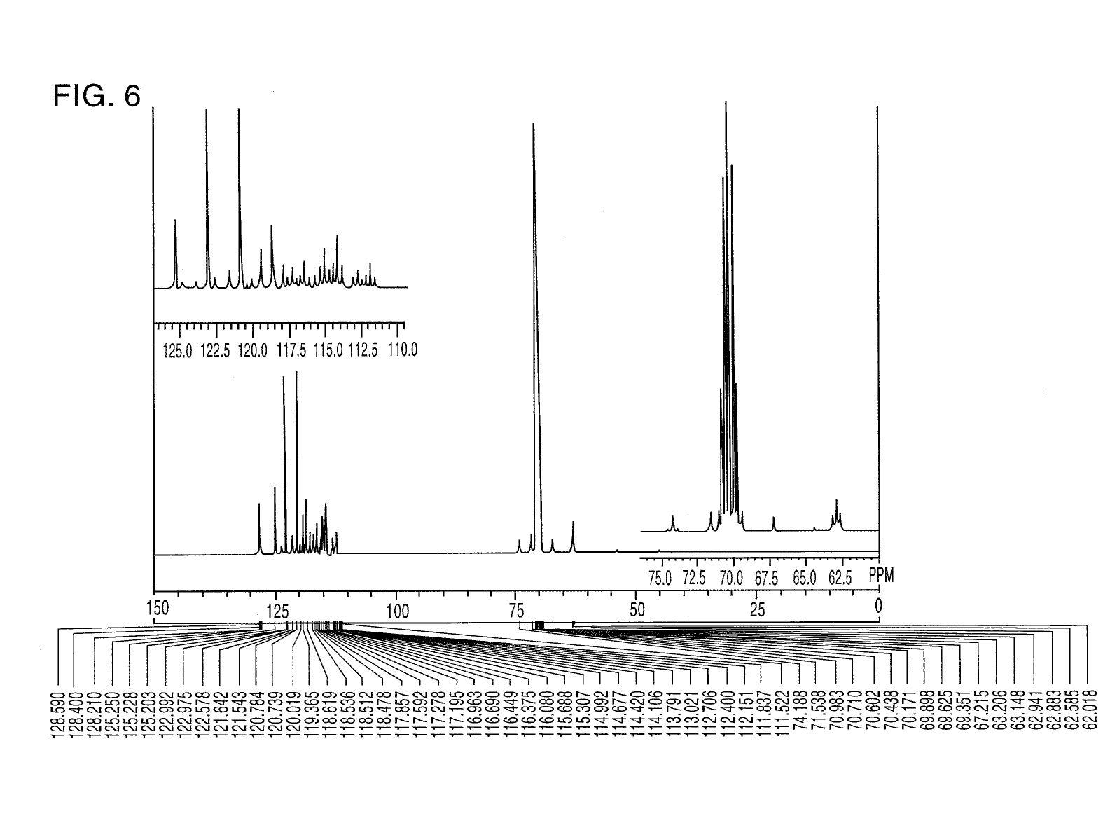

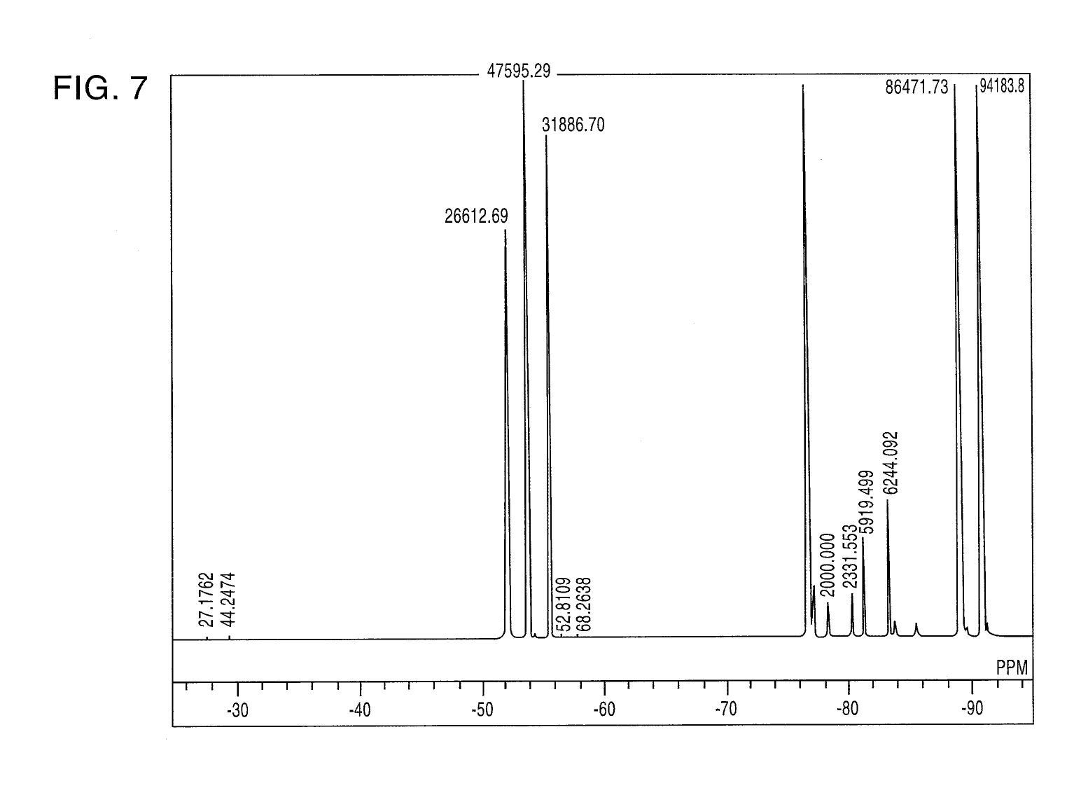

FTIR, ¹HNMR, ¹³CNMR, and ¹⁹FNMR were used to determine that this polymer had a molecular structure represented by formula 1 wherein n=a=b=0, and δ=1. FIGS. 5-7 show the obtained spectra. From ¹⁹FNMR, it was decided that the number average molecular weight was 2,273, the average degree of polymerization was 0.62, and the average value of the number average molecular weights of molecular chains (M1, M2, . . . ) between two adjacent hydroxy groups was 2,270. This number average molecular weight can be considered as the average value of the number average molecular weights (M1, M2, . . . ) that will be explained later.

Furthermore, the above-described polymer lubricant was subjected to dissolution and extraction, using a supercritical carbon dioxide fluid, and varying the temperature and the pressure. TABLE 1 summarizes the number average molecular weights (Mn), the average degree of polymerization (r), the number average molecular weights (Mc) of molecular chains (M1, M2, . . . ) between two adjacent polar groups (hydroxy groups) and the average numbers of hydroxy groups per molecule, together with the fraction names (Fr1-Fr6).

TABLE 1

| | Mn | r | Mc | OH/molecule |
|---|---|---|---|---|
| Fr1 | 2,009 | 1.1 | 1,640 | 2.1 |
| Fr2 | 3.782 | 1.7 | 2,020 | 2.7 |
| Fr3 | 6,374 | 2.6 | 2,250 | 3.6 |
| Fr4 | 9,119 | 3.6 | 2,330 | 4.6 |
| Fr5 | 11,556 | 4.7 | 2,230 | 5.7 |
| Fr6 | 12,805 | 6.0 | 1,910 | 7.0 |

### Example 2

(Measurement of One-Molecule Film Thickness)

The one-molecule film thickness can be determined from the terrace structure that comes to appear when a lubricant is flowing during the observation of a change, with the passage of time, of a cross-sectional profile of a lubricant film thickness with an ellipsometer, as is disclosed in X. Ma et al., Journal of Chemical Physics, 1999, Vol. 110, p. 3129 to 3,137.

The Fr4 lubricant was applied to a part of the protective layer of a hard disk by a dip method to observe the cross-sectional profile of the lubricant film thickness change with the passage of time. As a result, a terrace structure came to appear, and the film thickness of the terrace (that is, one-molecule film thickness) was determined to be 1.74 nm.

In the same way, it was 1.66 nm in the case of FOMBLIN Z DOL (molecular weight being 2,022). That is, it was found that the fluorine-containing polymer according to EXAMPLE 1 had a one-molecule film thickness of the same level, although the molecular weight was roughly twice as large as that of FOMBLIN Z DOL. It is considered to be because the polar groups of the lubricant are adsorbed in the direction towards the substrate.

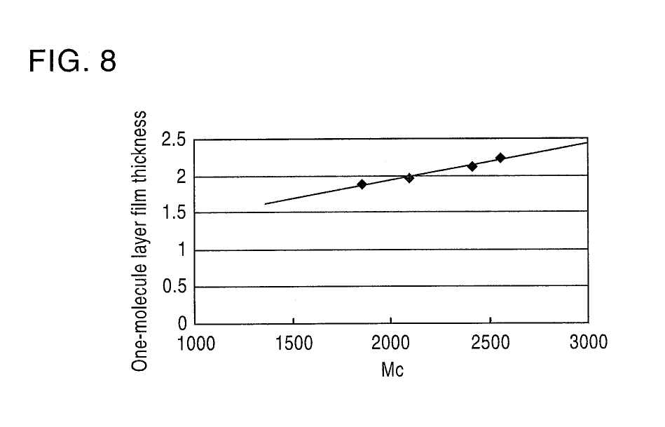

One-molecule layer film thicknesses obtained with the ellipsometer as described above were plotted against the number average molecular weights (Mc) of molecular chains (M1, M2, . . . ) between two adjacent polar groups (hydroxy groups) as shown in FIG. 8.

As a result, it was assumed that increasing the number average molecular weight to about 3,000, may make the one-molecule layer film thickness as large as 2.5 nm. Since it is usually considered appropriate that the thickness of a coating film is about 80% of the one-molecule layer film thickness, the thickness of a coating film is 2 nm when Mc is about 3,000, if such way of thinking is applied. It is often considered too large, in view of the contribution to the magnetic spacing and the head flying stability. Accordingly, it can be regarded more preferable that the range not more than 3,000 is appropriate from the viewpoint of Mc.

It is to be noted here that the coverage of the coating surface was considered sufficient in view of the results of measurement of the surface free energy. In other words, while the surface free energy of FOMBLIN Z DOL was 23 mN/m, it was 18 mN/m in the case of the Fr4 fluorine-containing polymer according to EXAMPLE 1, indicating that a sufficiently low surface energy state can be provided.

### Example 3

(Evaluation of Flying Properties)

The lubricant (Fr4) obtained in EXAMPLE 1 was applied to the protective layer of a hard disk by the dip method and subjected to a glide test for evaluating the flying properties. The coating thickness of the lubricant was made to be 1.2 nm. Heat treatment was carried out at an ambient temperature of 130° C. for 50 minutes. This examination monitors the output of a piezoelectric element, when a disk is made to rotate at a peripheral speed of 8.6 m/s and a slider equipped with the piezoelectric element is made to fly at a height of 6 nm.

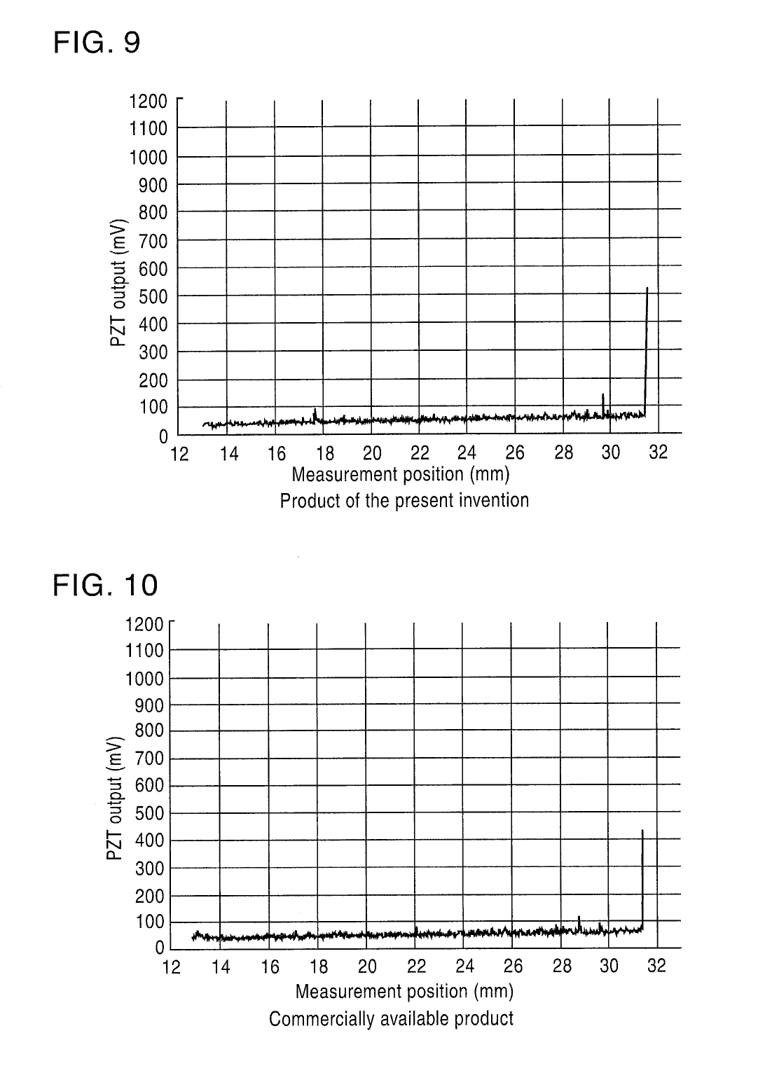

For the purpose of comparison, the same examination was carried out to a commercially available disk having a recording density of 38 Gbit/inch² (converted value being 5.89 Gbit/cm²). As shown in FIGS. 9 and 10, the results indicate that there was no difference between the two, and the magnetic disk using the lubricant according to the present invention for the lubricant layer had good flying properties. In FIGS. 9 and 10, the abscissa axis represents the location in the radial direction of a disk, and the axis of ordinate represents the output voltage of a piezoelectric element. The symbols A and B represent the surfaces of a disk.

Furthermore, for the purpose of comparison, FOMBLIN Z DOL (molecular weight being 4,000), a commercially available product, was applied to the protective layer of a hard disk by the dip method and subjected to a glide test for evaluating the flying properties. The coating thickness of the lubricant was made to be 1.2 nm. Heat treatment was carried out at an ambient temperature of 130° C. for 50 minutes.

It was found that the head did not fly under this condition. It is considered that flying failure occurred, because the molecular weight was large, and accordingly, the one-molecule layer film thickness was large.

### Example 4

(Evaluation of Flying Properties)

The take-off velocity (TOV) and touch-down velocity (TDV) were determined using a CSS (Contact-Start-Stop) testing machine. TOV is defined as the velocity at which a head starts to fly when the rotation number of a disk is increased, and TDV is defined as the velocity at which a head collides with a disk when the rotation number of the disk is decreased.

The head used was a head with a flying height of 10 nm. The lubricant (Fr4 in EXAMPLE 1) was used for a magnetic recording medium lubricant layer for the magnetic disk used. The thickness of the coating film was 1.2 nm. The same heat treatment as in EXAMPLE 3 was carried out.

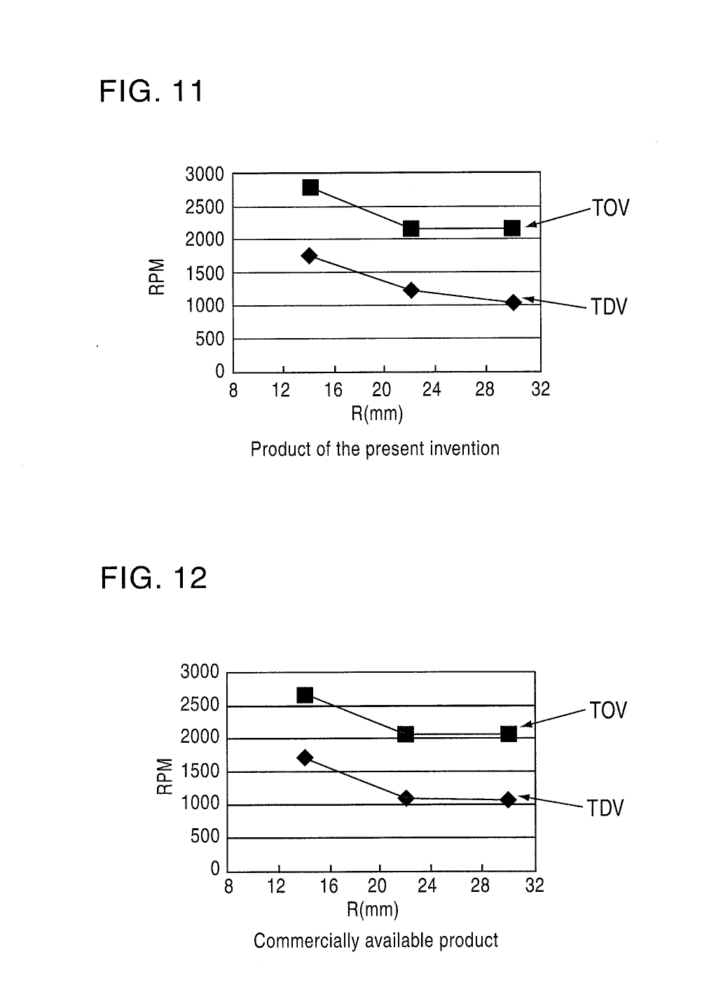

For the purpose of comparison, the same examination was carried out for a commercially available disk having a recording density of 38 Gbit/inch² (converted value being 5.89 Gbit/cm²). As shown in FIGS. 11 and 12, the results indicate that there was no difference between the two, and the magnetic disk using the lubricant according to the present invention had good flying properties. In FIGS. 11 and 12, the abscissa axis represents the location in the radial direction of a disk, and the axis of ordinate represents the rotation number of a disk.

What is claimed is:

1. A lubricant comprising a fluorine-containing polymer having a structure represented by formula 1,
H—{(XZ)_{m′}(YZ)ₙ}-(X_{δ},Y_{(1-δ)})—H (1)
[in formula 1, m and n are each a real number not less than zero (wherein m and n are not zero at the same time); {(XZ)ₘ, (YZ)ₙ} means that the structure unit XZ and the structure unit YZ may each take a random sequence or a blocked sequence; similarly, (X_{δ}, Y_{(1-δ)}) means that the structure unit X and the structure unit Y may each take a random sequence or a blocked sequence; δ is a real number not less than zero and not more than 1; and the chemical structures of X, Y and Z are as shown in formulae 12, 13 and 14 in this order, wherein hydrogens on X, Y and Z may be each substituted with an organic group having from one to three carbons that may contain an ether bond, and may have its hydrogen or hydrogens substituted with one or both of a polar group and fluorine,]
—O(CH₂CH₂O)ₐCH₂CF₂O(CF₂CF₂O)ₚ(CF₂O)_{q}CF₂CH₂ (OCH₂CH₂)_{b}O— (12)
{in formula 12, a, b, p and q are each a real number not less than zero (wherein p and q are not zero at the same time); and the structure units CF₂CF₂O and CF₂O may each take a random sequence or a blocked sequence,} 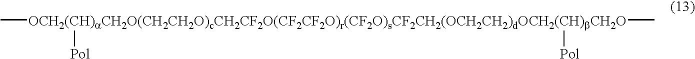
{in formula 13, c, d, r and s are each a real number not less than zero (wherein r and s are not zero at the same time); α and β are, independently from each other, a positive real number; the structure units CF₂CF₂O and CF₂O may each take a random sequence or a blocked sequence; and Pols are, independently from each other and independently from the other formulae, a polar group,} 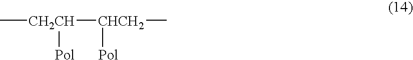
(in formula 14, Pols are, independently from each other and independently from the other formulae, a polar group).

2. A lubricant according to claim 1, wherein the number average molecular weight of molecular chains that are each between two adjacent polar groups of said fluorine-containing polymer is not less than 500.

3. A lubricant according to claim 1, wherein the number average molecular weight of said fluorine-containing polymer is not less than 2,000 and not more than 12,000.

4. A lubricant according to claim 1, wherein said Pol is a hydroxy group.

5. A lubricant according to claim 1, wherein the number average molecular weight of molecular chains that are each between two adjacent polar groups of said fluorine-containing polymer is not more than 3,000.

6. A lubricant according to claim 1, wherein said fluorine-containing polymer has a structure represented by formula 3 at its molecular end,
—R (3)
(in formula 3, R represents a polar group or a hydrocarbon group.)

7. A lubricant according to claim 1, wherein part or the whole of structure units C₂F₄O and CF₂O in formula 1 is substituted with a structure represented by formula 4 or a structure represented by formula 5.

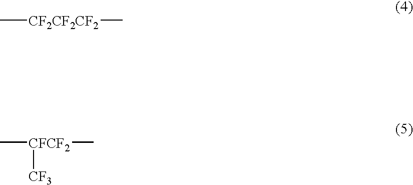

8. A lubricant according to claim 1, wherein no polar group is present except a hydroxy group in said fluorine-containing polymer.

9. A lubricant according to claim 1, wherein there are from 1.0 to 10.0 hydroxy groups per molecule on an average in the intermediate section of a molecular chain of said fluorine-containing polymer.

10. A lubricant comprising a fluorine-containing polymer obtained by reacting a compound having a structure represented by formula 2 and 1,3-butadiene diepoxide,
HO(CH₂CH₂O)ₓCH₂CF₂O(CF₂CF₂O)ₚ″(CF₂O)_{q″}CF₂CH₂(OCH₂CH₂)_{y}OH (2)
(in formula 2, p″, q″, x and y are, independently from each other, a real number not less than zero (wherein p″ and q″ are not zero at the same time); and the structure units CF₂CF₂O and CF₂O may each take a random sequence or a blocked sequence.)

11. A lubricant according to claims 10, wherein said fluorine-containing polymer is obtained by reacting the compound having a structure represented by formula 2 and 1,3-butadiene diepoxide, followed by a further reaction with glycidol.

12. A lubricant according to claim 10, wherein the number average molecular weight of said fluorine-containing polymer is not less than 2,000 and not more than 12,000.

13. A lubricant according to claim 10, wherein there are from 1.0 to 10.0 hydroxy groups per molecule on an average in the intermediate section of a molecular chain of said fluorine-containing polymer.

14. A lubricant according to claim 10, wherein said fluorine-containing polymer has a structure represented by formula 3 at its molecular end,
—R (3)
(in formula 3, R represents a polar group or a hydrocarbon group.)

15. A lubricant according to claim 10, wherein part or the whole of structure units C₂F₄O and CF₂O in formula 2 is substituted with a structure represented by formula 4 or a structure represented by formula 5.

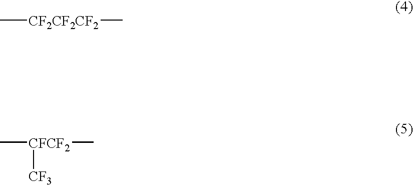

16. A lubricant according to claim 1, said lubricant comprising a fluorine-containing polymer other than said fluorine-containing polymer.

17. A lubricant according to claim 16, wherein the fluorine-containing polymer other than said fluorine-containing polymer is a compound having at least one structure selected from the group consisting of formulae 9, 10 and 11. 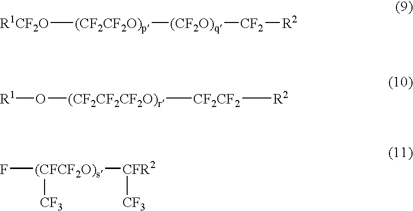
(in formulae 9 to 11, R¹ and R² are, independently from each other and independently in each formula, a group selected from the group consisting of a hydroxy group, a carboxy group, an amino group and a phosphazene ring, a monovalent aliphatic hydrocarbon group that has, as a substituent group or groups, one or more groups selected from the group consisting of a hydroxy group, a carboxy group, an amino group and a phosphazene ring, may have a carbonyl group, or an ether group, or a carbonyl group and an ether group, may have a double bond, or a triple bond, or a double bond and a triple bond, and may be branched, and a monovalent aromatic hydrocarbon group and a monovalent heterocyclic aromatic hydrocarbon group that comprise, as a substituent group or groups, one or more groups selected from the group consisting of a hydroxy group, a carboxy group, an amino group and a phosphazene ring; p′, q′, r′ and s′ are each a positive real number; and each structure unit of formulae 9, 10 and 11 may take a random sequence or a blocked sequence in the structure of each compound.)

18. A magnetic recording medium comprising a magnetic layer, a protective layer over said magnetic layer and a magnetic recording medium lubricant layer on said protective layer, said magnetic recording medium lubricant layer having been formed by the application of a lubricant according to claim 1.

19. A head slider having a recording transducer for recording to and/or reproducing information from a magnetic recording medium, comprising a protective layer and a head slider lubricant layer having been formed by the application of a lubricant according to claim 1, over the surface of the head slider that faces said magnetic recording medium.
